SEQUENCE LISTING (28 sequences)

SEQ ID NO: 1 - DNA, 20 bp, Artificial
  primer
acagggaagtgttccgtttg

SEQ ID NO: 2 - DNA, 20 bp, Artificial
  primer
ttgctgatcttcacccaatg

SEQ ID NO: 3 - DNA, 20 bp, Artificial
  primer
ctgcagcctcgagaagtagc

SEQ ID NO: 4 - DNA, 20 bp, Artificial
  primer
tcacacaccttgtcatcagg

SEQ ID NO: 5 - DNA, 20 bp, Artificial
  primer
tggtaagcagccttgtcctc

SEQ ID NO: 6 - DNA, 20 bp, Artificial
  primer
tggtaagcagccttgtcctc

SEQ ID NO: 7 - DNA, 20 bp, Artificial
  primer
actggcctaagctcatcctg

SEQ ID NO: 8 - DNA, 20 bp, Artificial
  primer
aggaggctgagggcattact

SEQ ID NO: 9 - DNA, 20 bp, Artificial
  primer
ttctcctggcttcttttcca

SEQ ID NO: 10 - DNA, 20 bp, Artificial
  primer
ttctcctggcttcttttcca

SEQ ID NO: 11 - DNA, 20 bp, Artificial
  primer
ttgggatcctctcagtcacc

SEQ ID NO: 12 - DNA, 20 bp, Artificial
  primer
ttgggatcctctcagtcacc

SEQ ID NO: 13 - DNA, 20 bp, Artificial
  primer
cagcaggtgtcacagaagga

SEQ ID NO: 14 - DNA, 20 bp, Artificial
  primer
atcctagcagtggagcagga

SEQ ID NO: 15 - DNA, 20 bp, Artificial
  primer
atcctagcagtggagcagga

SEQ ID NO: 16 - DNA, 20 bp, Artificial
  primer
cctggtctttccactggtgt

SEQ ID NO: 17 - DNA, 20 bp, Artificial
  primer
cctggtctttccactggtgt

SEQ ID NO: 18 - DNA, 20 bp, Artificial
  primer
cctggtctttccactggtgt

SEQ ID NO: 19 - DNA, 20 bp, Artificial
  primer
cctggtctttccactggtgt

SEQ ID NO: 20 - DNA, 20 bp, Artificial
  primer
ccaagatgcaatgatggatg

SEQ ID NO: 21 - DNA, 20 bp, Artificial
  primer
ccaagatgcaatgatggatg

SEQ ID NO: 22 - DNA, 20 bp, Artificial
  primer
acaccactagggagccacat

SEQ ID NO: 23 - DNA, 20 bp, Artificial
  primer
aggcctaatcgggaacttgt

SEQ ID NO: 24 - DNA, 20 bp, Artificial
  primer
aggcctaatcgggaacttgt

SEQ ID NO: 25 - DNA, 20 bp, Artificial
  primer
aggcctaatcgggaacttgt

SEQ ID NO: 26 - DNA, 20 bp, Artificial
  primer
aggcctaatcgggaacttgt

SEQ ID NO: 27 - DNA, 20 bp, Artificial
  primer
aaattggtggcttggctatg

SEQ ID NO: 28 - DNA, 20 bp, Artificial
  primer
aaattggtggcttggctatg

(19) United States
(12) Patent Application Publication
(10) Pub. No.: US 2010/0261183 A1
(43) Pub. Date: Oct. 14, 2010

(54) METHOD OF DETERMINING RISK FOR CANCER

(76) Inventors: Adam Marcus Shlien, Toronto (CA); David Daniel Malkin, Toronto (CA)

Correspondence Address: VALENTINE A COTTRILL;SUSAN TANDAN, 50 QUEEN STREET NORTH, STE. 1020, P.O. BOX 2248, KITCHENER, ON, N2H6M2, CA

(21) Appl. No.: 12/740,533

(22) PCT Filed: Oct. 31, 2008
(86) PCT No.: PCT/CA08/01920

§ 371 Date: Jun. 15, 2010

Related U.S. Application Data
(60) Provisional application No. 60/996,120, filed on Nov. 1, 2007.

(51) Int. Cl. C12Q 1/68

(52) U.S. Cl. 435/6

(57) ABSTRACT

A method of determining risk of cancer in a mammal is provided. The method includes analyzing the genomic DNA of the mammal and determining genomic CNV frequency or genomic structural variation. An increase in either CNV frequency or genomic structural variation in comparison to a baseline mean value is indicative of cancer.

### FIELD OF THE INVENTION

The present invention relates to the field of oncology, and in particular relates to a method of determining the risk of a mammal to develop cancer.

### BACKGROUND OF THE INVENTION

Cancer is an incremental process involving multiple changes at tumor suppressor and oncogenes. Common genetic variants, such as single nucleotide polymorphisms (SNPs), that modify or accelerate this process can contribute to early-onset tumors or familial aggregations of cancer. Acquired chromosomal changes are frequently found in tumor genomes, causing gene deletions, amplifications or balanced cytogenetic abnormalities and their importance in somatic tumorigenesis is well established. As with SNPs, constitutional deletions and duplications, such as CNVs, are recognized as important components of genetic variation.

A CNV is a segment of DNA 1 kb or larger that is present in variable copy number in the genomes of humans, primates and potentially many other species. A first-generation map of CNVs in the human genome was recently completed, revealing 1,447 variable regions in 270 individuals from the HapMap collection. Knowledge of frequency of CNVs per population is necessary for the characterization of rare disease-associated regions, while knowledge of the baseline number of CNVs per person will aid in identifying individuals with particularly unstable genomes.

The potential role of CNVs as genetic risk factors to cancer predisposition has not yet been explored. Accordingly, there is a need to explore the role of CNVs associated with risk of cancer.

### SUMMARY OF THE INVENTION

It has now been determined that an increased number of genomic CNVs in a mammal is indicative of risk of or predisposition for cancer.

Accordingly, in one aspect of the invention, a method of determining risk of cancer in a mammal is provided comprising the steps of:
determining in a genomic nucleic acid-containing sample obtained from the mammal the number of CNVs in the genome of the mammal, wherein an increase in the number of CNVs as compared to a baseline mean value is indicative of a risk of cancer in the mammal.

In another aspect of the present invention, a method of determining risk of cancer in a mammal is provided comprising the steps of:
(i) determining in a genomic nucleic acid-containing sample obtained from the mammal the structural variation in the genome of the mammal, wherein structural variation of at least about 1.1 megabases of DNA in comparison to a baseline value is indicative of risk of cancer.

These and other aspects of the present invention are described by reference to the following figures in which:

### BRIEF DESCRIPTION OF THE FIGURES

FIG. 1 illustrates the distribution of CNV frequencies in the normal population;

FIG. 2 illustrates by boxplots CNV frequency and total structural variation for 4 ethnic groups;

FIG. 3 illustrates the distribution of CNV frequencies in controls, TP53 wild type individuals and TP53 mutation carriers;

FIG. 4 is a bargraph of CNV frequency in controls, TP53 wild type individuals, TP53 mutation carriers unaffected by cancer and TP53 mutation carriers affected by cancer;

FIG. 5 is a comparison of copy number within the genomic DNA of a patient prior to onset of cancer and subsequent to onset of cancer; and

FIG. 6 is a boxplot illustrating CNV frequencies for TP53 wild type controls and TP53 carriers affected with cancer.

### DETAILED DESCRIPTION OF THE INVENTION

A method of determining risk of cancer in a mammal is provided. The method comprises determining in a genomic nucleic acid-containing sample obtained from the mammal the number of CNVs in the genome of the mammal. A determination of an increased number of CNVs in comparison to a baseline mean value is indicative of a risk of cancer in the mammal.

The term “CNV” is used herein to refer to copy number variations in genomic DNA, including both deletions and insertions of DNA, either partial genes, full genes, regions encompassing one or more genes or regions not encompassing any coding region in whole or in part.

The term “baseline mean value” refers to the mean number of CNVs which is expected to be present in the genome of a healthy mammal. The baseline mean, as one of skill in the art will appreciate, is the average of the number of CNVs in a group of healthy mammals.

The term “mammal” is used herein to refer to both human and non-human mammals. The term “healthy mammal” refers to a mammal in which there is no evidence of disease, and in particular, any type of cancer.

In the present method of determining risk of cancer in a mammal, a genomic nucleic acid-containing biological sample obtained from the mammal is utilized. Examples of suitable biological samples include saliva, urine, semen, other bodily fluids or secretions, epithelial cells, cheek cells, hair and the like. Although such non-invasively obtained biological samples are preferred for use in the present method, one of skill in the art will appreciate that invasively-obtained biological samples, may also be used in the method, including for example, blood including lymphoblasts, serum, bone marrow, cerebrospinal fluid (CSF) and tissue biopsies such as epithelial tissue. Techniques for the process of obtaining such samples are known to those of skill in the art.

To conduct the method of the present invention, a genomic nucleic acid-containing sample is obtained from a mammal being assessed. The sample is obtained from the mammal using methods conventional for the specific sample type and stored in a suitable manner until it is analyzed. The amount of sample required to conduct the assessment is an amount that is sufficient to allow identification of CNVs, for example, a minimum amount of about 500 ng of genomic DNA.

Prior to analyzing the sample, it may be necessary to process the sample to yield a form acceptable for analysis. For example, the nucleic acid (e.g. genomic DNA) may be extracted from the sample using techniques well-established in the art including chemical extraction techniques utilizing phenol-chloroform (Sambrook et al., 1989), guanidine-containing solutions, or CTAB-containing buffers. As well, as a matter of convenience, commercial DNA extraction kits are also widely available from laboratory reagent supply companies, including for example, the QIAamp DNA Blood Minikit available from QIAGEN (Chatsworth, Calif.), or the Extract-N-Amp blood kit available from Sigma (St. Louis, Mo.).

Once an appropriate sample is obtained, the DNA is genotyped using multiplexed microarray bead-based technology. In this regard, the sample is processed by restriction enzyme digestion, amplification, purification, labelling, fragmentation and hybridization, techniques all well-established in the art. DNA copy number may be determined using, for example, quantitative PCR.

A determination of an increased number of CNVs in comparison to a baseline mean value is indicative of a risk of or pre-disposition for cancer in the mammal. It will be appreciated that the baseline mean value may vary with a given population. It will also be appreciated that the absolute value of the increase in CNV frequency will vary depending on the resolution of the method utilized to determine CNV frequency. An increase in CNV frequency of at least about 1.2 times the baseline mean has been determined to indicative of risk for cancer, for example an increase in CNV frequency of about 1.5 times the baseline value or greater, such as 2-4 times the mean baseline value. In one embodiment, utilizing a resolution platform having for example, about 500,000 probes, a baseline mean CNV value was determined to be a value of less than 4, for example a value of about 2-3.5, and values above this mean baseline were determined to be indicative of a risk of cancer. Thus, generally, the occurrence of more than 4 CNVs in a genome was determined to be indicative of an increased risk of cancer. In another embodiment, utilizing a higher resolution platform (e.g. about 1.8 million probes), higher absolute values for baseline mean and CNV frequency in affected mammals was determined.

A determination of structural variation in the genome of a mammal in comparison to a baseline mean value may also be indicative of risk of cancer. The term “structural variation” is herein defined as the CNV frequency in a mammal multiplied by the average CNV size (in bp) in the mammal. Thus, high structural variation scores will result due to increased CNV frequency and/or due to the occurrence of large genomic nucleic acid deletions or duplications. This indicator is particularly relevant in connection with determination of cancer risk in mammals harbouring a TP53 mutation. A total structural variation score within genomic DNA of greater than about 1.1 megabases of DNA is indicative of risk of cancer.

The present method relates to the determination of risk of any cancer, including but not limited to, acute and chronic leukaemias, lymphomas, numerous solid tumors of mesenchymal or epithelial tissue, brain, breast, liver, stomach, colon cancer and other cancers linked to the TP53 mutations as described herein. In this regard, it is noted that the TP53 gene encodes the p53 transcription factor that functions as a tumor suppressor and, thus, is involved in blocking the transformation of normal cells to cancer cells. Mutations in the TP53 gene, such as in the DNA-binding domain (DBD) or in the homo-oligomerisation domain (OD), result in loss of function of p53 and loss of anti-cancer activity.

In another aspect of the invention, a method of diagnosing cancer in a mammal is also provided. In this regard, the determination in a biological sample obtained from a mammal of a CNV frequency of at least about 1.5 times the baseline mean CNV value may be indicative of cancer, for example a determination of 2-5 times the baseline mean CNV value, or even greater values, e.g. 5-10 times the baseline mean value. Generally, the CNV frequency is greater for a diagnosis of cancer in comparison to the CNV frequency that is indicative of risk of cancer as compared to a given baseline. As indicated above, absolute values will vary with the methods used to determine CNV frequency.

Embodiments of the present invention are described by reference to the following specific example which is not to be construed as limiting.

### Example 1

### Methods

Subject recruitment. After obtaining written informed consent, DNA was extracted from peripheral blood leukocytes of 53 individuals from families with a germline TP53 mutation and from 70 unrelated controls. This included 20 TP53 wild type and 33 TP53 mutation carriers. Of these, one individual had been diagnosed as a TP53 mosaic and was grouped with the TP53 mutation carriers in the CNV analysis. In addition, genomic DNA from 5 frozen choroid plexus tumors was extracted. DNA was quantified using a NanoDrop Spectrophotometer (NanoDrop, Wilmington, Del.) and quality assessed by agarose gel electrophoresis. This study was approved by the Research Ethics Board at the Hospital for Sick Children in Toronto. Subject recruitment for the 500 individuals of European descent and the 270 individuals from the HapMap collection are described elsewhere (Nature 437, 1299-320 (2005); Matsuzaki, H. et al. Nat Methods 1, 109-11 (2004)).

DNA microarray analysis and CNV determination. Genomic DNA was genotyped with Affymetrix GeneChip Human Mapping 500K Nsp and Sty arrays (Affymetrix, Santa Clara, Calif.); samples were restriction enzyme digested, amplified, purified, labeled, fragmented and hybridized as per the manufacturer's protocol. For the reference samples (n=770), DNA copy number analysis was performed with dChip as described (Lin, M. et al. Bioinformatics 20, 1233-40 (2004)) using Affymetrix Nsp CEL files. The LFS case-control cohort (n=123) was assessed with dChip, CNAG (Nannya, Y. et al. Cancer Res 65, 6071-9 (2005)) and GEMCA (Komura, D. et al. Genome Res 16, 1575-84 (2006)) using Affymetrix Nsp and Sty CEL files. Two samples with more than 150 CNVs were excluded from the TP53 mutation carrier group to avoid calling a high number of false positives.

Quantitative PCR validation. Quantitative PCR of genomic DNA copy number was performed by relative quantification on a Roche LightCycler 480 (Roche Applied Science, Indianapolis, Ind.) instrument using the Roche SYBR green kit. Primers were designed using Primer3 and the human genome reference assembly (UCSC version hg17, based on NCBI build 35). All samples were run in triplicate. Copy number alterations were assessed by relative quantification methods which compensate for differences in target and reference amplification efficiencies. Primer sequences are indicated below in Table 1. qPCR cycling conditions (repeated for 40 cycles): 95° C. for 10 seconds; 60° C. for 15 seconds; and 72° C. for 10 seconds, Preceded by 95° C. for 5 minutes. Tm is melting temperature.

Table 1
Quantitative PCR primers

| Primer name | Orientation | Sequence | Tm |
|---|---|---|---|
| 21q21.1 | Forward | 5-ACAGGGAAGTGTTCCGTTTG | 60 |
| | | (SEQ ID No: 1) | |
| 21q21.q | Reverse | 5-TTGCTGATCTTCACCCAATG | 60 |
| | | (SEQ ID No: 2) | |
| MLLT4 | Forward | 5-CTGCAGCCTCGAGAAGTAGC | 60 |
| | | (SEQ ID No: 3) | |
| MLLT4 | Reverse | 5-TCACACACCTTGTCATCAGG | 60 |
| | | (SEQ ID No: 4) | |
| 22q11.23-0 | Forward | 5-TGGTAAGCAGCCTTGTCCTC | 60 |
| | | (SEQ ID No: 5) | |
| 22q11.23-0 | Reverse | 5-ACACTGGCCCATCCCTTAG | 60 |
| | | (SEQ ID No: 6) | |
| 22q11.23-1 | Forward | 5-ACTGGCCTAAGCTCATCCTG | 60 |
| | | (SEQ ID No: 7) | |
| 22q11.23-1 | Reverse | 5-AGGAGGCTGAGGGCATTACT | 60 |
| | | (SEQ ID No: 8) | |
| CNV23rdm | Forward | 5-TTCTCCTGGCTTCTTTTCCA | 60 |
| | | (SEQ ID No: 9) | |
| CNV23rdm | Reverse | 5-ACCCTAAGCTCCTGCAGACA | 60 |
| | | (SEQ ID No: 10) | |
| CNV31rdm | Forward | 5-TTGGGATCCTCTCAGTCACC | 60 |
| | | (SEQ ID No: 11) | |
| CNV31rdm | Reverse | 5-GATTCCTGCCTTCCAATTCA | 60 |
| | | (SEQ ID No: 12) | |
| CNV47rdm | Forward | 5-CAGCAGGTGTCACAGAAGGA | 60 |
| | | (SEQ ID No: 13) | |
| CNV47rdm | Reverse | 5-ATCCTAGCAGTGGAGCAGGA | 60 |
| | | (SEQ ID No: 14) | |
| CNV87rdm | Forward | 5-CCATGTCTGTGGTGCTATGG | 60 |
| | | (SEQ ID No: 15) | |
| CNV87rdm | Reverse | 5-CCTGGTCTTTCCACTGGTGT | 60 |
| | | (SEQ ID No: 16) | |
| CNV110rdm | Forward | 5-CTGACTCAGGAGGCGATAGG | 60 |
| | | (SEQ ID No: 17) | |
| CNV110rdm | Reverse | 5-GTCCAACCCTTCACTTTCCA | 60 |
| | | (SEQ ID No: 18) | |
| CNV66rdm | Forward | 5-GCCACTCCCTTGTATGGAAA | 60 |
| | | (SEQ ID No: 19) | |
| CNV66rdm | Reverse | 5-CCAAGATGCAATGATGGATG | 60 |
| | | (SEQ ID No: 20) | |
| CNV120rdm | Forward | 5-TCTGTGTCCCCTGACTTTCC | 60 |
| | | (SEQ ID No: 21) | |
| CNV120rdm | Reverse | 5-ACACCACTAGGGAGCCACAT | 60 |
| | | (SEQ ID No: 22) | |
| CNV139rdm | Forward | 5-AGGCCTAATCGGGAACTTGT | 60 |
| | | (SEQ ID No: 23) | |
| CNV139rdm | Reverse | 5-CACCACCTACTGGGAGGGTA | 60 |
| | | (SEQ ID No: 24) | |
| CNV153rdm | Forward | 5-CCCTCTCCACTGTGCTTCTC | 60 |
| | | (SEQ ID No: 25) | |
| CNV153rdm | Reverse | 5-CTGTAAACACCTGCCCCACT | 60 |
| | | (SEQ ID No: 26) | |
| CNV160rdm | Forward | 5-AAATTGGTGGCTTGGCTATG | 60 |
| | | (SEQ ID No: 27) | |
| CNV160rdm | Reverse | 5-GCCTTTCACTTGAGCAGGTC | 60 |
| | | (SEQ ID No: 28) | |

Statistical analyses. Data was analyzed using SPSS versions 14.0 and 15.0 (SPSS Inc, Chicago, Ill.). CNV frequencies were natural logarithm transformed and compared by two-tailed independent-samples t-tests after assessing for normality using stem and leaf plots and histograms. A p-value of <0.05 was considered significant. Levene's test for equality of variances was used to determine when to assume equal variances. To compare the frequency of the cancer-related CNV overlapping MLLT4, the Fisher's exact test was used. Unrelated probands in the LFS cohort (n=19) were evaluated for the CNV and contrasted to unrelated individuals in the reference population (n=710, all children from the CEPH and Yoruban trios were excluded to ensure independent observations).

Computational assessment of cancer-related genes. Cancer-related genes were selected from the CancerGenes database (Higgins M E, et al. Nucleic Acids Res 35: D721-D726). Genes with zero sources were excluded, yielding a final list of ˜400 known cancer-related genes. Genomic coordinates of CNVs and genes were based on the NCBI build 35 reference human genome sequence. Custom software (available upon request) was used to determine CNVs encompassing or overlapping genes in more than one individual.

TP53 mutation screening. TP53 mutations were detected by direct sequencing of exons 2 to 11 and intron-exon boundaries of PCR products from blood-derived DNA using an ABI automated sequencer. Primer sequences used are known in the art (Tabori U, et al. Cancer Res 67:1415-1418, the contents of which are incorporated by reference).

### Results

### Characterization of Copy Number Variation

3,884 CNVs were identified in genomic DNA from 770 reportedly healthy individuals using Affymetrix GeneChip 500K Nsp microarrays. This cohort included 500 individuals of European descent and the multi-ethnic 270 person HapMap collection. The European cohort was analyzed on blood-derived DNA and the HapMap cohort on lymphoblastoid cell line derived DNA. Samples were grouped by microarray facility and normalized against members of their group to reduce batch effects. CNVs were then determined using dChip. To minimize false positives, CNVs on autosomal chromosomes comprised of 2 or more underlying single nucleotide polymorphism (SNP) probes only were counted.

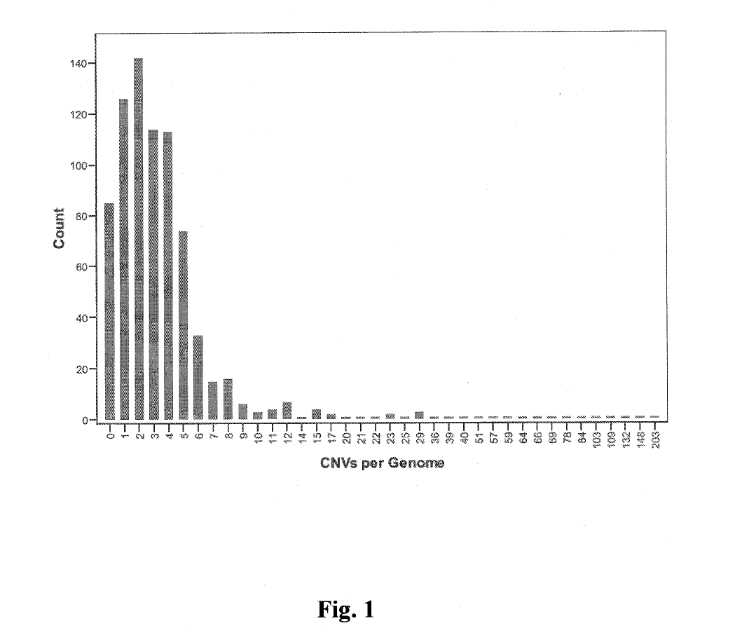

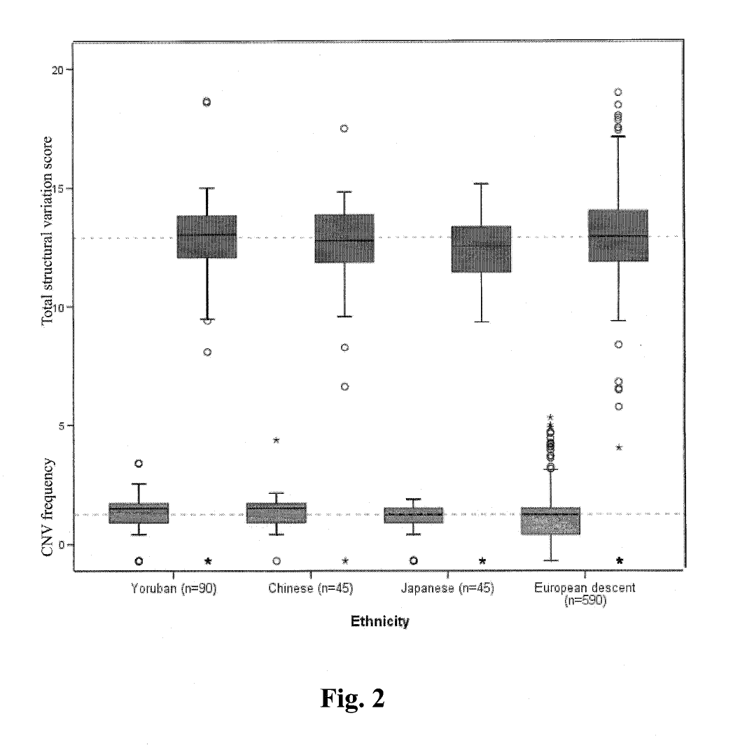

Many CNVs were found in single individuals while others, such as the CNV at chromosome 10q11.22 identified in 63 people, were found in numerous individuals, demonstrating the variability of the CNV population frequency. In contrast, the frequency of CNVs per genome appears to be highly conserved: the median number of CNVs detected per person was 3, with 75% of the population having 4 or fewer CNVs (FIG. 1). Moreover, CNV frequency appeared to be independent of ethnicity, as a separate analysis of the Yorubans, Chinese, Japanese and individuals of European descent revealed a similar result (FIG. 2). Despite conserved CNV frequencies, the varying size of these deletions and duplications could still result in individuals with different amounts of copy number-variable DNA. To investigate this possibility, a simple metric was created, termed total structural variation, defined as the CNV frequency multiplied by the individual's average CNV size (in bp). The median total structural variation showed a similar degree of conservation and was calculated to be 395 kb, with 75% of the healthy population having 1.1 Mb or less copy variable DNA (FIG. 2).

Having established the distribution and frequency of CNVs in a large reference population, deviations from the global norm in 11 well-characterized cancer predisposed LFS families were studied. Inherited TP53 mutations were observed in 9 families and de novo TP53 mutations in the other two families as shown in Table 2.

TABLE 2
LFS Families

| Families | TP53 mutation | n | WT | Mutation carriers |
|---|---|---|---|---|
| 1 | Arg175His | 3 | 1 | 2 |
| 2 | Arg273Ser | 4 | 2 | 2 |
| 3 | 12138 insC; pro72fs | 3 | 1 | 2 |
| 4 | Pro152Leu | 3 | 1 | 2 |
| 5 | Arg175His | 5 | 3 | 2 |
| 6 | Arg158His | 4 | 1 | 3 |
| 7 | IVS03-11 C > G | 6 | 2 | 4 |
| 8 | His193Pro | 4 | 3 | 1 |
| 9 | Phe134Tyr | 3 | 1 | 2 |
| 10 | Arg248Gln | 6 | 3 | 3 |
| 11 | Tyr163Cys | 4 | 3 | 1 |

Forty-five family members were evaluated. Eight additional unrelated TP53 mutation carriers were included for whom DNA samples were unavailable from other family members (Table 3). Of these 53 individuals, 33 were TP53 mutation carriers and 20 harbored wild type TP53.

TABLE 3
Unrelated TP53 mutation carriers

| Unrelated carriers | TP53 mutation |
|---|---|
| 1 | Arg248Gln |
| 2 | IVS05-1 G > C |
| 3 | c.652insG; Glu221Stop |
| 4 | Arg273His |
| 5 | Arg175His |
| 6 | 14494-1450 del8/ins AGGTG; Cys275Stop |
| 7 | Arg273Cys |
| 8 | Arg273His |

In addition, 70 unrelated healthy controls were evaluated for CNVs. Both Affymetrix GeneChip 250K Nsp and Sty microarrays were utilized for all analyses, and validation was performed using two additional CNV detecting algorithms.

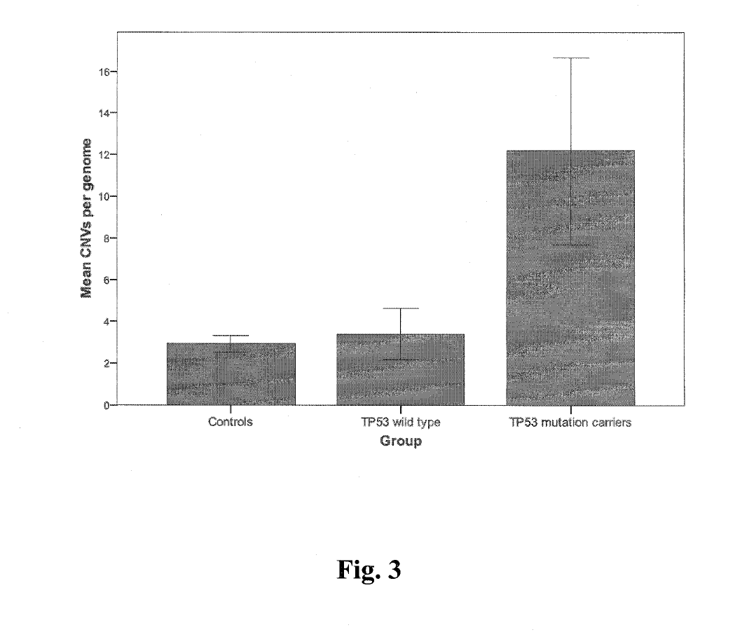

Similar to the large reference population, controls displayed a median of 2 CNVs per genome, with 75% of the population having 4 or fewer CNVs (mean=2.93). Additionally, no significant difference in CNV frequency between controls and the TP53 wild type group (median=2, 75^{th} percentile=3, mean=3.4) were detected. In contrast, the TP53 mutation carriers displayed a significant increase in CNVs (p=0.01). This cancer-prone group displayed a mean of 12.19 CNVs per genome with 75 percent having 10 or fewer CNVs (median=3, FIG. 3). Of the 33 carriers, 17 exhibited more alterations than the baseline. Remarkably, every LFS family with an inherited TP53 mutation, except one, contained individuals with CNV counts above the global norm. The majority of CNVs in LFS family trios were acquired (on average twice as common than inherited CNVs) and mutation carriers with a family history of cancer were significantly more likely to have an increase in CNVs when compared to their mutation carrier parent (p=0.015, Fisher's exact test, observed/expected ratios:2.0 for carriers and 0.0 for their wild-type siblings).

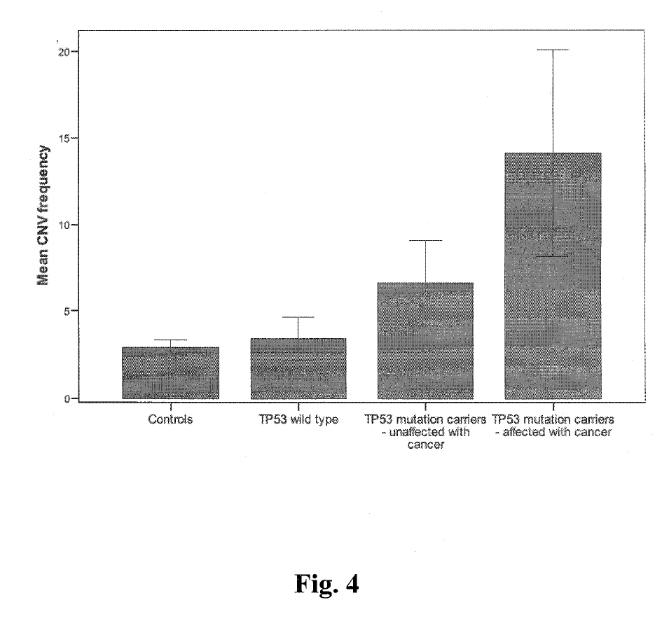

Eight of the eleven families studied had histories of cancer. The only families that did not have high CNV frequencies were those that did not have a family history of cancer. Of these, two had a single affected proband with a de novo TP53 mutation (Tyr163Cys and His193Pro). The other family had a single affected child who harbored an extremely rare paternally inherited TP53 mutation (Phe134Tyr). Many of the TP53 mutation carriers also had higher total structural variation scores than TP53 wild-type individuals, which is as one would expect given their numerous CNVs. Less anticipated were individuals found to have few CNVs but high total structural variation scores, as a consequence of exceptionally large deletions or duplications. The most dramatic example found was a paternally inherited 6.1 Mb deletion on chromosome 21 (21q21.1-q21.2) in an LFS family (FIG. 4). The deletion was confirmed by quantitative PCR (qPCR) of DNA derived from blood or normal paraffin-embedded tissue in the absence of available blood (p<0.01 in all cases). SNP genotypes were examined in the same region and a 6 Mb stretch of homozygosity was identified, which is as expected as the individual harboured only a single allele at this locus. Both affected children in this family harbored the deletion and a germline TP53 mutation (Arg273Ser, maternally inherited). The confluence of these two genetic events, high total structural variation and a germline TP53 mutation, thus correlates with the increase in cancer incidence observed in the family.

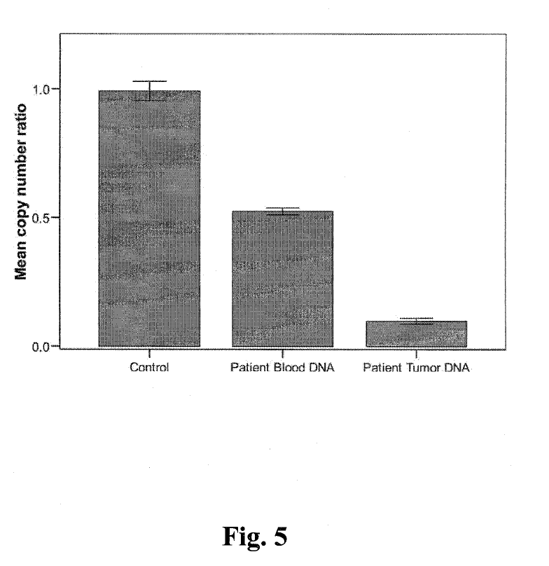

Increased CNV frequency was found by comparing individuals at elevated risk for cancer to those at normal risk (TP53 mutation carriers versus TP53 wild type individuals). Although nearly all mutant TP53 carriers will develop cancer in their lifetime, a determination of whether CNV frequency may also explain the clinical variability within the TP53 mutant (at-risk) group was desired. The CNV frequency of TP53 mutation carriers affected by cancer was examined separately from the unaffected carriers. The unaffected and affected groups each had significantly increased CNV frequencies as compared to controls (p=0.009 and p=0.046, respectively). Of particular interest is the presence of an even greater number of CNVs present in those affected by cancer, when compared to those who have not as yet developed cancer. These results indicate a dose-response relationship between CNV frequency and severity of the LFS phenotype (FIG. 5). Whether exposure to chemotherapy influences accumulation of germline structural alterations is not known. However, the fact that blood was drawn prior to starting therapy in almost all of the patients in this study, and the observation of increased germline CNVs even in those mutant TP53 carriers who are not yet affected with cancer, suggest that therapy does not contribute to accumulation of germline DNA structural variations.

The effect of germline CNVs on the development of somatic chromosomal alterations in paired tumor tissue was examined. DNA was extracted from five frozen tumor samples, taken from individuals whose constitutional CNVs were known, and hybridized on the same microarray platform. Choroid plexus tumours (choroid plexus carcinoma and choroid plexus papilloma) were selected since they frequently occur within the context of LFS. Several loci where germline hemizygous deletions progressed into homozygous deletions in the tumour or where germline duplications became further amplified in the tumour were noted. Because the presence of gross tumour chromosome changes could artificially inflate the observed number of such events, regions undergoing discrete changes localized to the underlying CNV were selected. One such CNV, a loss at 22q11.23, underwent an additional somatic deletion while the rest of the chromosome maintained diploidy. Paired blood-tumour analysis also revealed a deletion in the tumour sample, indicating that the deletion was located at the same locus and was expanded beyond that observed in the patient's blood. qPCR confirmed a one copy loss in the germline as compared to a diploid reference, and at the same locus, a one copy loss in tumour DNA as compared to the germline (FIG. 5). It therefore appears that germline CNVs can act as a basis for more dramatic tumour-specific changes.

### Example 2

In a reference population, which included 500 persons of European descent and the multiethnic 270 person HapMap collection, 49 cancer-related genes encompassed or directly overlapped by a CNV were identified as set out in Tables 4A and 4B below.

TABLE 4A
Most frequent cancer-related germline CNVs

| Gene | Gene name | RefSeq ID | N | Location |
|---|---|---|---|---|
| MLLT4 | Myeloid/lymphoid or mixed-lineage leukemia (trithorax | NM_005936 | 13 | 6q27 |
| | (trithorax homolog, Drosophila); translocated to, 4 | | | |
| FHIT | Fragile histidine triad gene | NM_002012 | 11 | 3p14.2 |
| TFG | TRK-fused gene | NM_006070 | 7 | 3q12.2 |
| FANCF | Fanconi anemia, complementation group F | NM_022725 | 6 | 11p15 |
| MSH6 | mutS homolog 6 (E. coli) | NM_000179 | 6 | 2p16 |
| CENPK | Centromere protein K | NM_022145 | 4 | 5q12.3 |
| MAML2 | Mastermind-like 2 (Drosophila) | NM_032427 | 4 | 11q |
| POT1 | POT1 protection of telomeres 1 homolog (S. pombe) | NM_015450 | 4 | 7q31.33 |
| RAD51L1 | RAD51-like 1 (S. cerevisiae) | NM_133510 | 4 | 14q23-q4.2 |
| RPS6KA2 | Ribosomal protein S6 kinase, 90 kDa, polypeptide 2 | NM_021135 | 4 | 6q27 |

TABLE 4B
Additional cancer-related germline CNVs

| Gene | | | | |
|---|---|---|---|---|
| Symbol | Gene Name | RefSeq ID | Num | Location |
| ABL1 | v-abl Abelson murine leukemia viral oncogene homolog 1 | NM_007313 | 3 | 9q34.1 |
| BCL10 | B-cell CLL/lymphoma 10 | NM_003921 | 3 | 1p22 |
| ERCC2 | Excision repair cross-complementing rodent repair | NM_000400 | 3 | 19q13.3 |
| | deficiency, complementation group 2 (xeroderma | | | |
| | pigmentosum D) | | | |
| FIP1L1 | FIP1 like 1 (S. cerevisiae) | NM_030917 | 3 | 4q11-q12 |
| FNBP1 | Formin binding protein 1 | NM_015033 | 3 | 9q34 |
| MDS1 | Myelodysplasia syndrome 1 | NM_004991 | 3 | 3q26 |
| MLF1 | Myeloid leukemia factor 1 | NM_022443 | 3 | 3q25 |
| PDGFRA | Platelet-derived growth factor receptor, alpha polypeptide | NM_006206 | 3 | 4q12 |
| RAP1GDS1 | RAP1, GTP-GDP dissociation stimulator 1 | NM_021159 | 3 | 4q23-q25 |
| AFF1 | AF4/FMR2 family, member 1 | NM_005935 | 2 | 4q21.3 |
| AFF4 | AF4/FMR2 family, member 4 | NM_014423 | 2 | 5q31 |
| BCL11A | B-cell CLL/lymphoma 11A (zinc finger protein) | NM_138559 | 2 | 2p16.1 |
| CDC73 | Cell division cycle 73, Paf1/RNA polymerase II complex | NM_024529 | 2 | 1q25 |
| | component, homolog (S. cerevisiae) | | | |
| CEBPA | CCAAT/enhancer binding protein (C/EBP), alpha | NM_004364 | 2 | 19q13.1 |
| CHN1 | Chimerin (chimaerin) 1 | NM_001822 | 2 | 2q31-q32.1 |
| CXXC6 | CXXC finger 6 | NM_030625 | 2 | 10q21 |
| ERCC5 | Excision repair cross-complementing rodent repair | NM_000123 | 2 | 13q22-q34 |
| | deficiency, complementation group 5 (xeroderma | | | |
| | pigmentosum, complementation group G (Cockayne | | | |
| | syndrome)) | | | |
| ETV6 | ets variant gene 6 (TEL oncogene) | NM_001987 | 2 | 12p13 |
| EVI1 | Ecotropic viral integration site 1 | NM_005241 | 2 | 3q26 |
| EXT1 | Exostoses (multiple) 1 | NM_000127 | 2 | 8q24.11 |
| FANCC | Fanconi anemia, complementation group C | NM_000136 | 2 | 9q22.3 |
| FGFR1OP | FGFR1 oncogene partner | NM_194429 | 2 | 6q27 |
| GAS7 | Growth arrest-specific 7 | NM_201433 | 2 | 17p13.1 |
| GPHN | Gephyrin | NM_020806 | 2 | 14q23.3 |
| IL2 | Interleukin 2 | NM_000586 | 2 | 4q26-q27 |
| JAZF1 | JAZF zinc finger 1 | NM_175061 | 2 | 7p15.2-p15.1 |
| KRAS | v-Ki-ras2 Kirsten rat sarcoma viral oncogene homolog | NM_033360 | 2 | 12p12.1 |
| LHFP | Lipoma HMGIC fusion partner | NM_005780 | 2 | 13q12 |
| MALT1 | Mucosa associated lymphoid tissue lymphoma | NM_173844 | 2 | 18q21 |
| | translocation gene 1 | | | |
| MDM2 | Mdm2, transformed 3T3 cell double minute 2, p53 | NM_006882 | 2 | 12q13-q14 |
| | binding protein (mouse) | | | |
| MLLT3 | Myeloid/lymphoid or mixed-lineage leukemia (trithorax | NM_004529 | 2 | 9p22 |
| | homolog, Drosophila); translocated to, 3 | | | |
| NOTCH1 | Notch homolog 1, translocation-associated (Drosophila) | NM_017617 | 2 | 9q34.3 |
| NR4A3 | Nuclear receptor subfamily 4, group A, member 3 | NM_173200 | 2 | 9q22 |
| PCM1 | Pericentriolar material 1 | NM_006197 | 2 | 8p22-p21.3 |
| PIK3CA | Phosphoinositide-3-kinase, catalytic, alpha polypeptide | NM_006218 | 2 | 3q26.3 |
| REL | v-rel reticuloendotheliosis viral oncogene homolog | NM_002908 | 2 | 2p13-p12 |
| | (avian) | | | |
| SS18 | Synovial sarcoma translocation, chromosome 18 | NM_005637 | 2 | 18q11.2 |
| TGFBR2 | Transforming growth factor, beta receptor II (70/80 kDa) | NM_003242 | 2 | 3p22 |
| WT1 | Wilms tumor 1 | NM_024426 | 2 | 11p13 |

Shown are cancer-related genes found to be directly overlapped, or fully encompassed by a germline CNV. For each gene, the number of individuals from the reference population harboring the CNV is indicated. In Table 4A, the most common genes are those present in greater than 3 apparently healthy individuals. Table 4B shows additional cancer-related CNVs present in 2 or 3 individuals.

In this study only the genes observed to be directly interacting with a CNV in more than one person were reported and, on this basis, 98 singular genes were excluded from the analysis. The current catalogue of genes implicated in cancer was obtained from the CancerGenes database and the CNV regions were determined from the oligonucleotide SNP array hybridizations (Higgins et al. Nucleic Acids Res 35, D721-6 (2007)). The most frequent copy number variable cancer genes observed were: MLLT4 (Myeloid/lymphoid or mixed-lineage leukemia [trithorax homolog, Drosophila] translocated to, 4); FHIT (Fragile histidine triad gene); TFG (TRK-fused gene); FANCF (Fanconi anemia, complementation group F) and MSH6 (mutS homolog 6 [E. coli]). These 49 copy number variable genes have been implicated in acute and chronic leukaemias, lymphomas and numerous solid tumors of mesenchymal or epithelial tissue.

The presence of apparently healthy individuals with CNVs at MSH6 were noted. Germline point mutations and gross genomic rearrangements at MSH6, MSH2, MLH1 and PMS2 are associated with Lynch Syndrome (or HNPCC), the most common form of inherited colorectal cancer. The FHIT gene was also determined to be the site of CNVs in this analysis. FHIT spans 1.5 Mb of DNA, encompasses the FRA3B fragile site and its protein is partially or entirely lost in most human cancers.

The LFS cohort also showed copy number variability in cancer-related genes. Of the nine families with inherited TP53 mutations assessed for CNVs, 2 families had near identical duplications on chromosome 6 (locus 6q27), overlapping the MLLT4 gene. MLLT4 is a target of Ras and is fused with MLL in the common leukemia translocation t(6;11)(q27;q23). The MLLT4 duplication was validated by qPCR in all individuals and in DNA from independent blood-redraws when available. The duplication was structurally similar to the CNV in the reference population (n=770): it's average size is 260 kb (min: 220 kb; max: 350 kb) in LFS and 250 kb (min: 240 kb; max: 372 kb) in the reference population. However, the frequency of the CNV is significantly increased in LFS (p=0.006, Fisher's exact test): Three of the 19 LFS probands (15.8%; Observed/Expected: 3/0.4=7.5) harbored the duplication, while only 12 of 710 healthy individuals from the reference population (1.69%; observed/expected: 12/14.6=0.82) harbored the CNV.

Another LFS family displayed two separate duplications on chromosome 10, which were inherited through three generations of family members. One of these duplications, at locus 10q26.2, intersects with the disintegrin-metalloproteinase ADAM12. The dysregulation of ADAM12 appears to be linked to cancers such as brain, breast, liver, stomach and colon cancers.

### Example 3

Genomic DNA was extracted from patient blood samples using the standard phenol-chloroform method. Briefly, for each sample, 500 nanograms of genomic DNA was digested with Nsp I and Sty I restriction enzymes and ligated to adaptors. Fragments ranging from 200 to 1100 basepairs were amplified, purified, fragmented, labeled and hybridized on Affymetrix Human 6.0 GeneChip microarrays, a higher resolution platform than that utilized in Example 1. Microarrays were then washed, stained and scanned.

Array probe signal intensities were normalized and then CNVs were determined using a binary genomic segmentation informatics algorithm. CNVs (deletions or duplications) in regions with too few probes (<10) or with insufficient probe coverage (<1 probe per 5000 bp) were excluded. To avoid a high false positive rate, individuals with greater than 1000 CNVs were omitted.

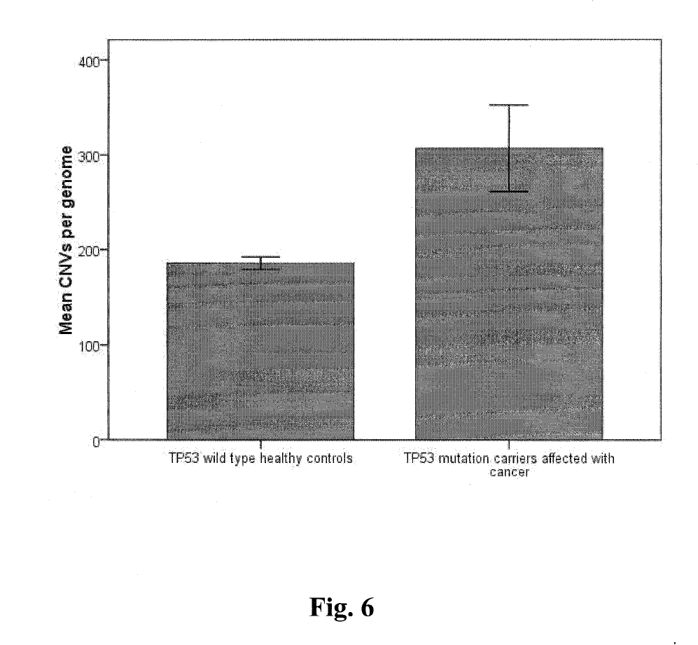

FIG. 6 illustrates the CNV frequencies of TP53 wild type healthy controls (n=149) and TP53 mutation carriers affected with cancer (n=21). A significant increase in CNVs was observed in those individuals affected with cancer, relative to healthy controls (a mean of 306.95 CNVs versus 186.05 CNVs, respectively). Error bars represent SEM.

### Platform Resolution

The studies described in Example 1 and Example 3 were performed using two different platforms, which differed in resolution. The higher-resolution platform (Affymetrix 6.0, described in Example 3) has over 1.8 million probes and an inter-marker distance of less than 700 basepairs, whereas the previous generation platform (Affymetrix 500k, described in Example 1) contained 500,000 probes with an inter-median probe distance of 2.5 Kb. The analysis using two different platforms demonstrates that the CNV frequency is demonstrably higher in TP3 mutation carriers affected with cancer than in healthy controls. It is noted that given the resolution differences between the platforms employed herein, the absolute CNV count differs from platform to platform.

### Discussion

The work presented herein establishes that risk of cancer and cancer diagnosis is linked to copy number variable regions and total structural variation. The results obtained from the LFS cohort can be extended to cancer in general because TP53 mutations, the most frequent genetic alteration in LFS, are the most commonly acquired genetic alteration in sporadic human cancer.

1. A method of determining risk of cancer in a mammal comprising the step of:
determining in a genomic nucleic acid-containing sample obtained from the mammal the number of CNVs in the genome of the mammal, wherein an increase in the number of CNVs in the genome of the mammal as compared to a baseline mean value is indicative of a risk of cancer in the mammal.

2. A method as defined in claim 1, wherein an increase in the number of CNVs in the genome of a mammal of at least about 1.2 times the baseline mean value is indicative of risk of cancer in the mammal.

3. A method as defined in claim 2, wherein an increase in the number of CNVs of at least about 2 times the baseline mean value is indicative of risk of cancer.

4. A method as defined in claim 3, wherein an increase in the number of CNVs in the range of about 2 to 4 times the baseline mean value is indicative of risk of cancer.

5. A method of determining risk of cancer in a mammal comprising the step of:
determining in a genomic nucleic acid-containing sample obtained from the mammal the structural variation in the genome of the mammal, wherein an increase in genomic structural variation in comparison to a baseline value is indicative of risk of cancer.

6. A method as defined in claim 4, wherein a determination of a genomic structural variation of at least about 1.1 megabases is indicative of risk of cancer.

7. A method of diagnosing cancer in a mammal comprising the step of:
determining in a genomic nucleic acid-containing sample obtained from the mammal the number of CNVs in the genome of the mammal, wherein an increase in the number of CNVs in the genome of the mammal as compared to a baseline mean value is indicative of cancer in the mammal.

8. A method as defined in claim 7, wherein an increase in the number of CNVs in the genome of a mammal of at least about 1.5 times the baseline mean value is indicative of cancer in the mammal.

9. A method as defined in claim 7, wherein an increase in the number of CNVs of at least about 2 times the baseline mean value is indicative of cancer.

10. A method as defined in claim 7, wherein an increase in the number of CNVs in the range of about 5 to 10 times the baseline mean value is indicative of cancer.
